(19) United States
(12) Patent Application Publication
(10) Pub. No.: US 2005/0208080 A1
(43) Pub. Date: Sep. 22, 2005

(54) Formulation for the administration of medicinal substances

(76) Inventors: Nicholas John Heightman, KENT (GB); Michael John Desmond Gamlen, Kent (GB)

Correspondence Address: KLAUBER & JACKSON, 411 HACKENSACK AVENUE, HACKENSACK, NJ, 07601, US

(21) Appl. No.: 10/501,808

(22) PCT Filed: Jan. 23, 2003
(86) PCT No.: PCT/GB03/00301

§ 371 Date: May 12, 2005

(30) Foreign Application Priority Data
Jan. 24, 2002 (GB) 0201607.9

(51) Int. Cl. A61K 9/00

(52) U.S. Cl. 424/400

(57) ABSTRACT

A formulation for administration of a medicinal substance, comprises a sandwich biscuit having two or more biscuit layers that support filler layer(s), in which the filler layer comprises a dosage unit form, or a multiple or sub-multiple thereof, of a medicament that is unpalatable in having, for example, a gritty texture or a chalky texture or other unpleasant mouth feel. The biscuit layer of the sandwich biscuit may be a plain, non-medicated, biscuit layer or may itself contain a medicament. In the latter instance, it is possible to select a different medicament for the filler layer from that in the biscuit layer, whereby the two medicaments have a co-operating or synergistic effect. The formulations also allow large dosages of drugs to be administered effectively in a palatable form and are suitable for the long-term administration of drugs.

### FIELD OF INVENTION

This invention relates to a new formulation for the administration of medicinal substances within traditional baked food products, in particular within sandwich biscuits.

### BACKGROUND OF THE INVENTION

Many medicines are potent materials requiring small amounts for their effectiveness, but there are other medicines that must be administered in large doses. Conventional formulations such as tablets and capsules can accommodate up to 1000 mg of active ingredient but the products are very large and many patients find them difficult to swallow. In many cases the physical properties of the medicinal substance preclude dosages in excess of 250-500 mg as they require dilution in inert materials to render them suitable for processing. Dosages of the order of several or many grammes per day require the patient to take many tablets. Some medicines such as guar gum and cholestyramine resin are presented in sachets for dispersion in water. The products are not very palatable and are inelegant, again resulting in problems with patient acceptability and compliance.

It is known that medicines can be made more palatable or their presence disguised by incorporating the medicines within pre-cooked biscuits that have been reduced to crumb form. However, this requires intervention on behalf of the person making up the mixture and relies on their skill in ensuring that both a full dose of medicine is incorporated within the mixture and that the patient consumes all of the mixture. It is also known that certain therapeutic substances can be incorporated within biscuits during the initial cooking step, but this is not always satisfactory, particularly if the incorporated substance is adversely affected by the cooking process. There thus remains a need to provide alternative formulations for unpalatable medicinal, i.e. pharmaceutically active, substances.

It is the purpose of the present invention to allow the inclusion of active medicinal substances in readily acceptable formulations in such a way that compliance with drug treatment dosage regimes in enhanced. The present invention also addresses the problem of allowing large dosages of drugs to be administered effectively, especially as it has often not previously been convenient to administer such dosages by known administration routes. Many treatment regimes achieve sub-optimal therapeutic results because patients for whom the treatment is prescribed find that it is unpleasant to take the drug in the amount required and accordingly the patient may either omit taking doses or take them in inadequate quantities. The present invention permits the administration of large amounts of unpalatable material—for example with a gritty texture or a chalky texture or other unpleasant mouth feel, in a product which both palatable and familiar to the patient. This helps to ensure acceptability to the patient, and thereby improve patient compliance.

We have found that the formulation according to the present invention can be adapted to carry relatively high quantities of medicinal drug substances and combination of medicinal substances in such a way that chewing in the mouth facilitates swallowing without adversely affecting the taste or mouthfeel of the biscuit. This makes the administration of drugs much more acceptable to many patients who find it difficult to swallow conventional pills and capsules. A further advantage is that formulations of the present invention have a texture that masks unpleasant mouth feel, such as gritty texture or chalkiness of some medicinal substances. The new formulations are also particularly suitable for the long-term administration of medicinal substances.

### SUMMARY OF THE INVENTION

According to the present invention there further provided a formulation for the administration of a medical substance comprising a sandwich biscuit having two or more biscuit layers that support filler layer(s), in which the filler layer, and optionally the biscuit layers, comprise(s) a dosage unit form, or a multiple or sub-multiple thereof, of an unpalatable medicament. A further aspect of the invention is that the filler layer can contain a large amount of medicinal substance without having a deleterious effect on the mouth feel or palatability of the product.

The “sandwich biscuit” of the present invention may comprise a cream or other filling layer supported between any convenient number of dry layers, normally two layers, of biscuit. The biscuit layer of the sandwich biscuit may be a plain, non-medicated, biscuit layer or may itself contain a medicament. In the latter instance, it is possible to select a different medicament for the filler layer from that in the biscuit layer, whereby the two medicaments have a co-operating or synergistic effect. As already indicated, a medicine is unpalatable if it cannot be readily orally administered in its simple state, for example because of unpleasant mouth feel.

As indicated previously, the invention is of special value where relatively large amounts of active medicinal ingredient need be taken for the treatment to be effective, for example an individual dose in excess of 250 mg, particularly in excess of 500 mg. Large amounts of medicament includes quantities between 1 g and 3 g per portion, although even higher quantities may be used in some circumstances. This feature of the invention can be particularly useful in both human and veterinary medicine.

The biscuits of the present invention would normally be subjected to a single cooking process and are otherwise known as cookies. Originally biscuits were subjected to two baking steps with the biscuit being dried in the second step. A rusk or similar product such as ships biscuits would be the result. Although such biscuits are long lasting, the second cooking step would normally be avoided in the present invention to prevent thermal damage to the medicinal compound and to make the product more palatable. Nevertheless, special technical steps must be taken to preserve the freshness of biscuits of the present invention as a considerably longer shelf life is contemplated for pharmaceuticals. For example, pharmaceuticals are often stored for up to 18 months, whereas baked products are normally consumed within a few weeks at the most. Appropriate steps for the long-term preservation of medical biscuits according to the invention might be the incorporation of suitable preservative substances or the use of specialised packaging materials to prevent the ingress of moisture and oxygen. Of course there may be occasions when it would be appropriate to store the medicated filling separately and arrange for the formation of the sandwich biscuit close to the anticipated date of consumption.

The sandwich biscuit embodiment of the present invention in which the medicament is only in the filling layer would be adopted if the medicament is heat sensitive and cannot be incorporated in the biscuit composition during the cooking stage without degradation.

The biscuits of the present invention would normally be prepared within food factories where the existing strict hygiene and quantity monitoring would be easily adapted to the strict requirements of the pharmaceutical industry. The various techniques for biscuit making on the domestic scale are generally adaptable to factory scale production. The main criterion for the suitability of a particular technique is the need to ensure that the weight and thus dosage of the individual portions of the resulting cooked biscuits can be tightly controlled. Examples of appropriate techniques are dropped cookies where soft dough is spooned directly on to the baking sheet in predetermined portions, shaped cookies where a predetermined portion is cut from soft dough, piped cookies where a relatively fluid mixture is piped onto a baking tray and bar cookies, such as flapjacks, in which a large block is produced and then cut after cooking into suitably sized portions.

Biscuit formulations can be prepared from a number of conventional ingredients and it is possible to select particular ingredients on the basis of their appropriateness to the disease to be treated. For example, if cholesterol reduction were the object of the therapy it would be possible to base a biscuit on a formulation on oats, which is a material that is believed to have additional therapeutic effect in lowering cholesterol. Such a biscuit layer would then be used in conjunction with a cholesterol lowering medicament in the filling layer.

In one embodiment the present invention accordingly provides a sandwich biscuit in which the medicinal substance in the biscuit and the medicament in the filler layer have a co-operating or synergistic effect on administration.

For the treatment of renal failure, sandwich biscuits contain an ion exchange resin substance VML252 with, or without a combination of calcium carbonate in the filler layer, or in both filler and biscuit layers, would be appropriate. This formulation would be used to treat elevated blood phosphate levels encountered by patients undergoing renal dialysis. This is one of the instances mentioned previously in which very high dosages of medicines are required and are difficult to present to the patient in an acceptable form in other ways. The presence of the medicament in both layers would be particularly appropriate in this instance. A typical dosage for the administration of such ion exchange resins would be approximately 8-12 g/day. Other treatments for hyperphosphataemia such as lanthanum carbonate and sevelamer hydrochoride (Renagel).

As already indicated, lowering cholesterol levels is also a possible treatment and sandwich biscuits for this purpose may contain the ion exchange resin, cholestyramine with, or without, other active substances including chlofibrate, gemfibrozil and other orally active cholesterol-lowering materials. Again high dosages may be necessary and a dosage of approximately 12 g/day of cholestyramine is typical.

Worm control in pets and farm animals may be based on sandwich biscuits formulated comprising anthelmintic agents for example, albendazole, febendazole (Panacura®, Ivermectin, thiabendazole and other bendazole substances. Appropriate version of such anthemintic formulations also may be used in human medicine.

For the treatment of diabetes type 2, sandwich biscuit formulation containing metformin or combinations of other oral agents with metformin would be appropriate, as would biscuit formulations with gamma guanidinobutyramide and its pharmaceutically acceptable salts together with combinations of other agents used to treat diabetes type 2. Appropriate dosages of these active ingredients would be 200 to 2000 mg.

Where a patient suffers from excessive serum potassium, sandwich biscuit formulations containing the ion exchange resin and combinations of other oral agents used for treating elevated serum potassium would be appropriate.

The following examples are provided to further illustrate the present invention

### EXAMPLE 1

A semi-sweet biscuit enriched with calcium carbonate was prepared from the following ingredients:

| | |
|---|---|
| Plain flour | 250 g |
| Sugar | 50 g |
| Margarine | 40 g |
| Salt | 3 g |
| Calcium carbonate | 53 g |
| Water to mix | 110 g |

The flour, salt and calcium carbonate were mixed together and the margarine rubbed in until the mixture resembled fine breadcrumbs. Water was added to form a firm dough, which was rested for 15 minutes. The dough was rolled to a thickness of about 2 mm and cut into circles weighing 7.5 g, so that each baked biscuit contained approximately 1 g of calcium carbonate.

The biscuits were baked on a greased baking sheet in a pre-heated oven at 190° C. for 5 minutes until a pale golden brown colour. The biscuits were then removed from the oven and allowed to cool.

The cream-filling was prepared using a cream made from the following ingredients:

| | | |
|---|---|---|
| Icing sugar | 237 | g |
| Fat | 240 | g |
| Lecithin | 0.6 | g |
| Salt | 0.3 | g |
| Vanilla liquid essence | 1.8 | g |
| Calcium carbonate | 120 | g |

The ingredients were mixed together to form a cream filling of which 5 g, containing 1 g calcium carbonate, was deposited between two semi-sweet biscuits, prepared as described above. This provided a total dose of 3 g calcium carbonate per biscuit. The biscuits were stored in an airtight container.

### EXAMPLE 2

A digestive biscuit enriched with calcium carbonate was prepared from the following ingredients:

| | |
|---|---|
| Plain flour | 160 g |
| Wheatmeal flour | 45 g |
| Margarine | 65 g |
| Caster sugar | 15 g |
| Demerara sugar | 35 g |
| Golden syrup | 15 g |
| Calcium carbonate | 30 g |
| Salt | 2 g |
| Water | 50 g |

The margarine, sugars and syrup were creamed together for 3 minutes and the dry ingredients folded into the resultant mixture. Water was added to form a firm dough, which was rested for 15 minutes. The dough was rolled to a thickness of about 3 mm and cut into circles weighing 14 g, so that each baked biscuit contained approximately 1 g of calcium carbonate.

The biscuits were baked on a greased baking sheet in a pre-heated oven at 185° C. for 6 minutes until a golden brown colour. The biscuits were then removed from the oven and allowed to cool.

A cream filling prepared as described in Example 1 was deposited between two biscuits. This provided a total dose of 3 g calcium carbonate per biscuit. The biscuits were stored in an airtight container.

### EXAMPLE 3

A semi-sweet biscuit containing an ion exchange resin VML 252 was prepared from the following ingredients:

| | | |
|---|---|---|
| Flour | 500 | g |
| Sugar | 104 | g |
| Fat | 80 | g |
| Glucose | 6.5 | g |
| Salt | 6 | g |
| Sodium bicarbonate | 2.5 | g |
| Ammoniuum bicarbonate | 5 | g |
| VML 252 | 107 | g |
| Water to mix | 220 | g |

The dough was mixed until a temperature of 40° C. was reached. The VML 252 was added to the dough mix and mixed until evenly distributed.

The dough was rested for 15 minutes before being formed into sheets of a thickness of 3 mm and cut into circles weighing 7.5 g, so that each baked biscuit contained approximately 1 g of VML 252.

The biscuits were baked on a greased baking sheet in a pre-heated oven at 190° C. for 5.5 minutes until a pale golden brown colour. After being removed from the oven and allowed to cool, the biscuits were stored in an airtight container.

The cream-filling was prepared using a cream made from the following ingredients:

| | | |
|---|---|---|
| Icing sugar | 237 | g |
| Fat | 240 | g |
| Lecithin | 0.6 | g |
| Salt | 0.3 | g |
| Vanilla liquid essence | 1.8 | g |
| VML 252 | 120 | g |

The ingredients were mixed together to form a cream filling. 5 g of the filling containing 1 g VML 252 was deposited between two semi-sweet biscuits, prepared as described in Example 5. This provided a total dose of 3 g VML 252 per biscuit. The biscuits were stored in an airtight container.

### EXAMPLE 4

A low salt snack cracker was prepared from the following ingredients:

| | |
|---|---|
| Fat (reduced fat spread | 160 g |
| rich in monounsaturates) | |
| Salt | 13 g |
| Skimmed milk powder | 16 g |
| Flour | 800 g |
| Ammonium bicarbonate | 20 g |
| Sodium metabisulphite | 10 g |
| Water | 450 g |

The ingredients were added in the above order and blended together for 1 minute. The dough was then mixed at 120 rpm for 3.5 minutes. The dough was rested for 15 minutes before being formed into sheets and laminated to a final thickness of 2 mm.

The dough was cut into circles weighing 8 g. The crackers were baked at 230° C. for 4.5 minutes. After being removed from the oven, the crackers were allowed to cool.

A cream cheese filling containing gamma guanidinobutyramide (an antidiabetic agent) was prepared using a cream made from the following ingredients:

| | |
|---|---|
| Fat (reduced fat spread | 240 g |
| rich in monounsaturates) | |
| Lecithin | 0.6 g |
| Cheese flavouring | 2.0 g |
| Gamma guanidinobutyramide | 2.0 g |

The cream filling was added between two biscuit layers in an amount that provided 200 to 2000 mg of gamma guanidinobutyramide per sandwich biscuit. The biscuits were then stored in an airtight container.

### EXAMPLE 5

A low salt snack cracker was prepared as described in Example 4.

A cream cheese filling containing metformin (an antidiabetic agent) was prepared using a cream made from the following ingredients:

| | |
|---|---|
| Fat (reduced fat spread | 240 g |
| rich in monounsaturates) | |
| Lecithin | 0.6 g |
| Cheese flavouring | 2.0 g |
| Metformin | 2.0 g |

The cream filling was added between two biscuit layers in an amount that provided 200 to 2000 mg of metformin per sandwich biscuit. The biscuits were then stored in an airtight container.

### EXAMPLE 6

Semi sweet biscuits were prepared as described in Example 1, but with the omission of the calcium carbonate component.

A biscuit cream filling was prepared by creaming together the following ingredients:

| | |
|---|---|
| Icing sugar | 100 g |
| Vegetable shortening | 60 g |
| Lanthanum carbonate | 40 g |
| Lemon/vanilla flavouring | trace |

A 5 g portion of the cream filling was sandwiched between two semi-sweet biscuits. Each cream biscuit provided 1 g lanthanum carbonate.

### EXAMPLE 7

A serving of medicated biscuit was prepared from two ‘morning coffee’ biscuits sandwiched together with 10 g cream containing 3 g Renagel® (sevelamer hydrochloride).

The Renagel® cream filling was prepared by blending together the following components to form a soft cream:

| | |
|---|---|
| Icing sugar | 40 g |
| Vegetable shortening | 30 g |
| Renagel | 30 g |
| Lemon flavouring | trace |

The filling and biscuits, respectively had the following nutritional properties:

| Filling nutritional composition | per 100 g | per 10 g serving |
|---|---|---|
| Energy | 1782 kJ; 428 kcal | 178 kJ; 43 kcal |
| Protein | 0 g | 0 g |
| Carbohydrate (as sugars) | 42.0 g | 4.2 g |
| Fat | 30.0 g | 3.0 g |
| Saturates | 6.3 g | 0.6 g |

Nutritional composition of each cream biscuit

| | |
|---|---|
| Energy | 368 kJ; 89 kcal |
| Protein | 0.8 g |
| Carbohydrate | 11.6 g |
| Sugars | 6.0 g |
| Fat | 4.4 g |
| Saturates | 1.2 g |
| Sodium | less than 0.1 g |

It will, of course, be understood that the present invention has been described above purely by way of example and that modifications of detail can be made within the scope of this invention.

1. A formulation for the administration of a medicinal substance as used in compliance with a drug treatment dosage regime comprising a sandwich biscuit comprising two or more biscuit layers that support layers wherein the cream filling layer comprises a dosage unit form or multiple or sub-multiple thereof of an unpalatable medicament.

2. A formulation according to claim 1, wherein the medicament has a gritty texture or a chalky texture or other unpleasant mouth feel.

3. A formulation according to claim 1 wherein the medicament is present in an amount of greater than 500 mg per biscuit.

4. A formulation according to claim 3, wherein the medicament is present in an amount of between 1 g and 3 g per biscuit.

5. A formulation according to claim 1 wherein the medicinal substance is selected from the ion exchange resin substance VML252, optionally in combination of calcium carbonate, the ion exchange resin cholestyramine, optionally in combination chlofibrate, gemfibrozil and other orally active cholesterol-lowering materials, anthelmintic agents, metformin or gamma guanidinobutyramide and its pharmaceutically acceptable salts, optionally in combination of other oral agents used to treat diabetes type 2, optionally in combination with other agents for the oral treatment of obesity, and ion exchange resin suitable for treating elevated serum potassium, optionally in combination of with other oral agents used for treating elevated serum potassium.

6. A formulation according to claim 5, wherein the anthelmintic agent is selected from the group consisting of albendazole, febendazole, Ivermectin, thiabendazole and another bendazole substances.

7. A formulation according to claim 1 wherein the biscuit layers comprise a medical substance, wherein the medicinal substance in the biscuit and the medicament in the cream filling layer have a co-operating or synergistic effect on administration.

8. A formulation according to claim 1 wherein the biscuit layer is an oatmeal biscuit and the medicament is a cholesterol lowering pharmaceutical.

9. (canceled)
